(19) United States
(12) Patent Application Publication
(10) Pub. No.: US 2003/0083237 A1
(43) Pub. Date: May 1, 2003

(54) Anti-angiogenic peptides for cancer treatment

(76) Inventors: Dexian Dou, Livonia, MI (US); Jiajiu Shaw, Ann Arbor, MI (US)

Correspondence Address: Jiajiu Shaw, 3166 Shamrock Ct., Ann Arbor, MI, 48105, US

(21) Appl. No.: 09/927,006

(22) Filed: Aug. 9, 2001

(51) Int. Cl. A61K 38/00

(52) U.S. Cl. 514/9

(57) ABSTRACT

This invention discloses two peptides and their derivatives that are anti-angiogenic and method for treating cancer and other diseases related to angiogenesis.

### FIELD OF INVENTION

This invention relates to anti-angiogenic peptides for treating cancer and other diseases caused by the abnormal growth of blood vessels.

### BACKGROUND

Angiogenesis is a phenomenon wherein new blood vessels are created to form a new vascular network in the living body. The inner wall of blood vessel is comprised of endothelial cells, thus; the angiogenesis varies depending on the function and growth of endothelial cells.

Most tumor cells rely on new blood vessels to supply the necessary nutrition. Therefore, inhibiting the growth of new blood vessels around cancerous area can be effective in treating cancer. This area of research has also become popular for treating other diseases resulted from abnormal growth of new blood vessels.

The abnormal growth of new capillary blood vessels can also cause other disease, such as inflammation of joint membrane. Vision impairment of older people and late staged diabetes patients may also be resulted by angiogenesis. It is believed that anti-angiogenesis is the way to treat this type of diseases.

Without any blood vessels, the tumor usually can only grow to 1˜2 μM, which is not life threatening. Many times, the metastasis of cancer (e.g., lung cancer) is caused by the formation of new capillary blood vessels on the existing vessels around the tumor area. The endothelial cells on the inside of these new capillary vessels are stimulated by cancer cells and begin the abnormal growth. Cancer cells are thus supplied with more nutrition through these new capillary vessels and begin to grow at a fast speed. Because of the fast growth, there is a local deficiency of oxygen (hypoxia) on the inside of the tumor. This hypoxia environment causes the cancer cells to generate stimulants, such as VEGF(vascular endothelial growth factor) or bFGF (basic fibroblast growth factor). These stimulants again stimulate the growth of yet other new blood vessels, which then supply more nutrition to the tumor cells. These cyclic reactions cause the continuous growth of the tumor.

There are several known proteins having anti-angiogenic activities including thrombospondin (Kosfeld N M et al, J. Bio. Chem., 267, 16230-16236 (1993); Tolsma S S et al, J. Cell Bio., 122, 497-509 (1993); Dameron K M et al, Science, 265, 1582-1584 (1994)), protamine (Arrieta O et al, Eur. J. Cancer, 34, 2101-2106 (1998)), angiostatin (O'Reilly M et al, Cell, 79, 315-328 (1994), laminin (Sakamoto N et al, Cancer Res., 51, 903-906 (1991)), endostatin (O'Reilly M et al, Cell, 88, 277-285 (1997)), and a prolactin fragment (Clapp C et al, Endocrinology, 133, 1292-1299 (1993)).

Several inhibitors or factors inhibiting the growth of endothelial cells have been used in treating diseases caused by or related to angiogenesis (Gasparini G et al, Journal of Clinical Oncology, 13, 765-782 (1995)). However, toxicity was observed, such as Suramin and Thelidomide. Some compounds only exhibit slight inhibition on the growth of endothelial cells, such as interferon and Tamoxifen.

Newer molecules such as Angiostatin are not easy to be manufactured by bioengineering technology.

Since some of the currently know anti-angiogenic agents cause toxic side effects, thereby posing problems in terms of safety. Some do not show sufficient antioangiogenic effects. In addition, the cost of producing anti-angiogenic protein drugs can be prohibitively high.

Therefore, it is of great significance and importance if a new and effective anti-angiogenic agent having low toxicity and low production costs can be found.

### DESCRIPTION OF THIS INVENTION

This invention discloses peptides and related technologies in the area of anti-angiogenesis. Compared to proteins, peptides are smaller and easier to manufacture, thus. More economic to produce. Therefore, it has been one of our goals to screen and discover anti-angiogenic peptide originated from human body.

The peptides disclosed in the present invention are of low molecular weights. In addition, said peptides in this invention are originated from human body, thus, much less likely to incur immune response or other side effects.

The following discloses the process and results of said anti-angiogenic peptides from human Apolipoprotein, Apo(a).

Peptides and proteins are comprised of amino acids. Between two amino acids, there is a peptide bond formed by a carboxyl group (—COOH) and an amino group (NH₂). Therefore, the polymer form by amino acids through peptide bonds is called peptide. There are numerous natural peptides in the cells with different lengths and compositions, resulting in different biological reactions associated with all kinds of cell activities. Usually, the longer peptides form various 3-dimensional shapes are called protein. In general, a synthetic process can produce proteins having up to about 80 amino acids. Larger proteins are usually produced by biological expressions.

Apo(a), is a large glycoprotein existing in normal human body (Mooser V et al, J. Clin. Invest., 97, 858-864 (1996). It biological function is not yet totally understood. However, its amino acids sequence has been. Within this huge Apo(a) molecule, there are 38 kringle structures. These kringles have different amino acid compositions and orders and may be categorized into 11 types.

The kringle structure was first discovered in thrombin. As used herein, “kringles” refers to a protein sequence with approximately 90 amino acids containing 6 cysteines that form 3 disulfide bridges. The basic function of a kringle structure is to mediate the intermolecular reactions. For example, t-PA (tissue-type plasminogen activator) consists of two kringle structures. The kringle near the C-terminal is capable of recognizing and combining fibrin in the blood clots, thus, providing the functions of targeting and dissolving the blood clots.

Within the 38 kringles of Apo(a), only one kringle was identified to be anti-angiogenic active (Dou D. et al., unpublished results). This anti-angiogenic kringle is located at the C-terminal of the protein, or the 38^{th} kringle from Apo(a) primary amino acid sequence. This 38th kringle was named as AK38. The DNA sequence encoding AK38 protein has been registered in the GeneBank under an access number of AY039748. GeneBank will publicly release AY039748 on Jul. 1, 2002.

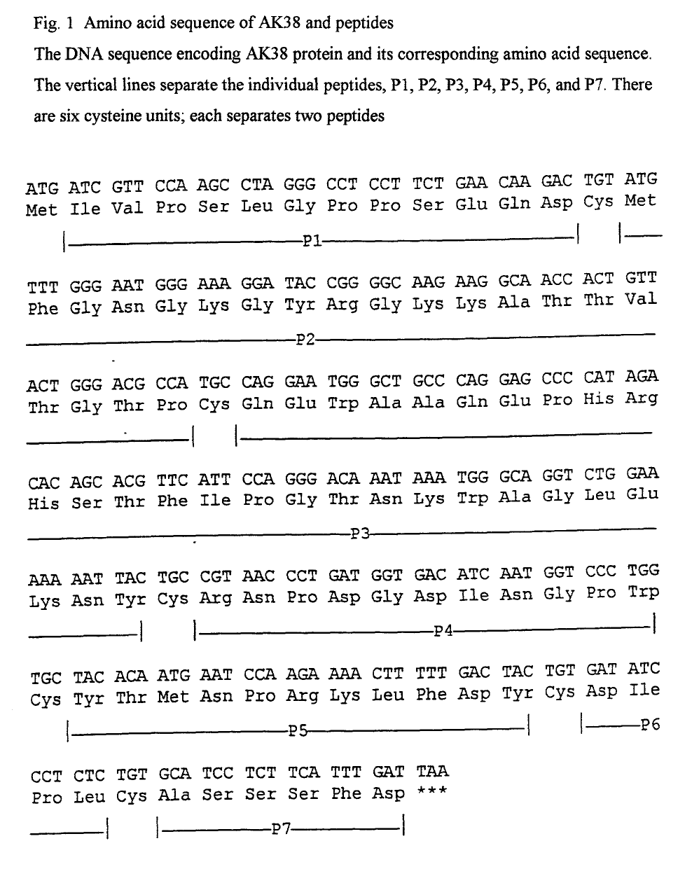

Kringle AK38 of Apo(a) is a typical kringle. Its primary amino acid sequence is shown at FIG. 1. Said anti-angiogenic peptides are portions of the AK38 protein. Said peptides disclosed in this invention were synthesized using Fmoc(9-fluorenylmethyloxycarbonyl)-based solid-phase peptide synthesis. HPLC method using C-18 column was used to purify the peptides. After purification, mass spectroscopy was used to identify the peptides.

The inhibition of said peptides on the endothelial cells was performed on BCE cells (Bovine Capillary Endothelial cells) as shown in Example 9.

The compositions of the seven peptides in AK38 kringle are listed as follow (C-terminus on the left and N-terminus on the right):

| | |
|---|---|
| {1} | Ile-Val-Pro-Ser-Leu-Gly-Pro-Pro-Ser-Glu-Gln- |
| | Asp |
| {2} | Met-Phe-Gly-Asn-Gly-Lys-Gly-Tyr-Arg-Gly-Lys- |
| | Lys-Ala-Thr-Thr-Val-Thr-Gly-Thr-Pro |
| {3} | Gln-Gln-Trp-Ala-Ala-Gln-Glu-Pro-His-Arg-His- |
| | Ser-Thr-Phe-Ile-Pro-Gly-Thr-Asn-Lys-Trp-Ala- |
| | Gly-Leu-Glu-Lys-Asn-Tyr |
| {4} | Arg-Asn-Pro-Asp-Gly-Asp-Ile-Asn-Gly-Pro-Trp |
| {5} | Tyr-Thr-Met-Asn-Pro-Arg-Lys-Leu-Phe-Asp-Tyr |
| {6} | Asp-Ile-Pro-Leu |
| {7} | Ala-Ser-Ser-Ser-Phe-Asp |

Each of these peptides (represented by {1}, {2}, {3}, {4}, {5}, {6}, and {7} is different in its length and composition. However, these peptides do have one thing in common: they are all located between two cysteine units within the kringle structure. For the experiments disclosed herein, each peptide is protected by acetyl group (CH₃CO—) at the N-terminus and by amino group (—NH₂) at the C-terminus, and is identified as P1, P2, P3, P4, P5, P6, and P7.

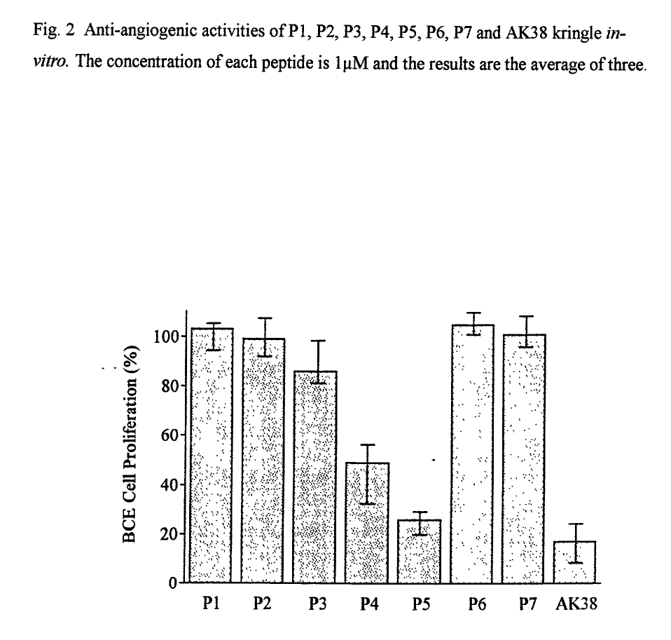

The in-vitro inhibition studies indicate that none of the four peptides (P1, P2, P6, and P7) is significantly effective. Peptide P3 did show minor inhibition effect. Surprisingly, P4 and P5 are very effective in inhibiting the growth of BCE cells (FIG. 2).

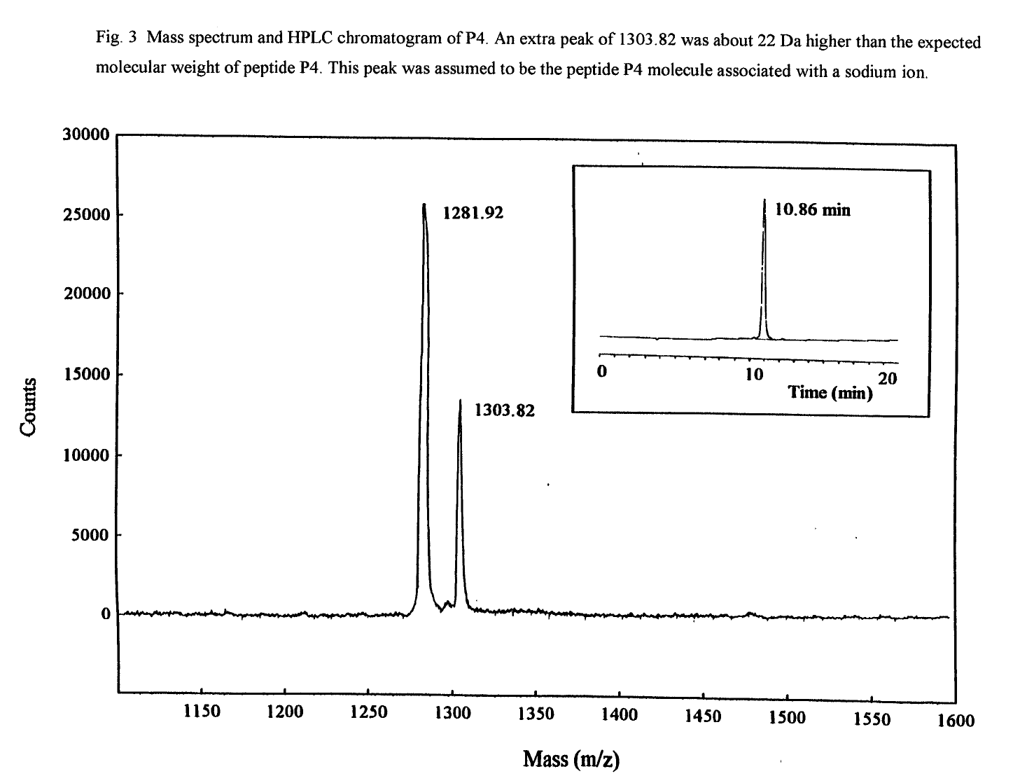

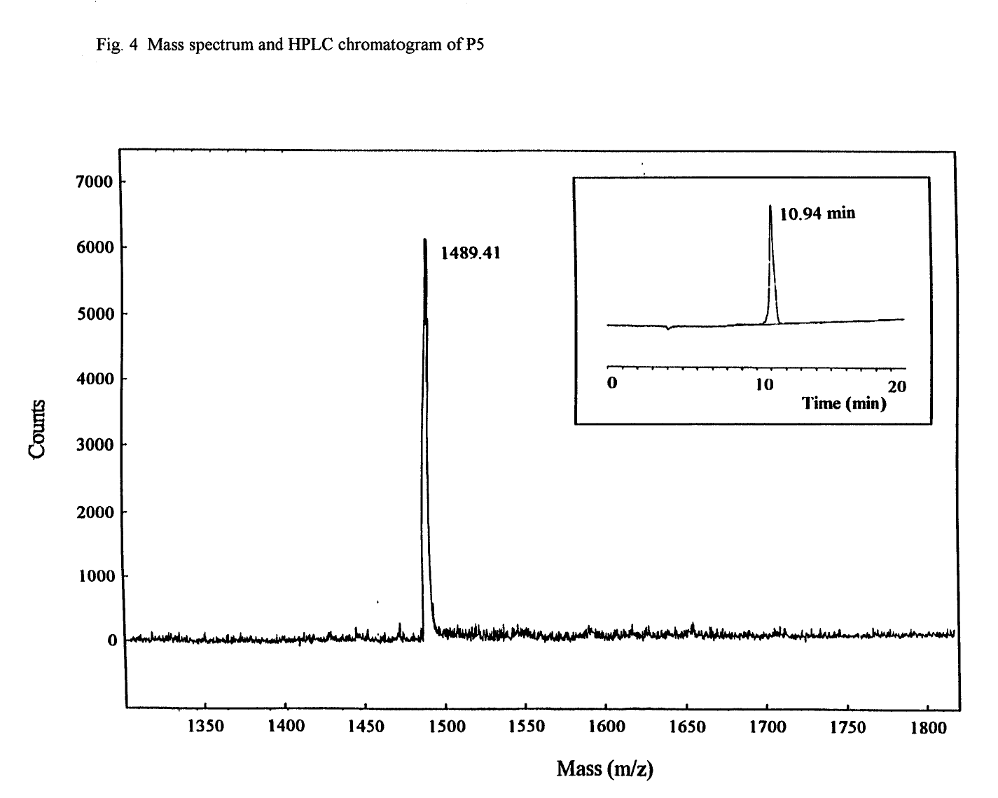

The theoretical molecular weight of P4 is of 1281.31 Da and that of P5 is 1488.66 da. The experimental data obtained by mass spectroscopy is 1281.92 Da for P4 and that of P5 is 1489.41 Da (FIG. 3 and FIG. 4). An extra peak of 1303.82 appeared at FIG. 3 was about 22 Da higher than the expected molecular weight of peptide P4. This peak was assumed to be the peptide P4 molecule associated with a sodium ion.

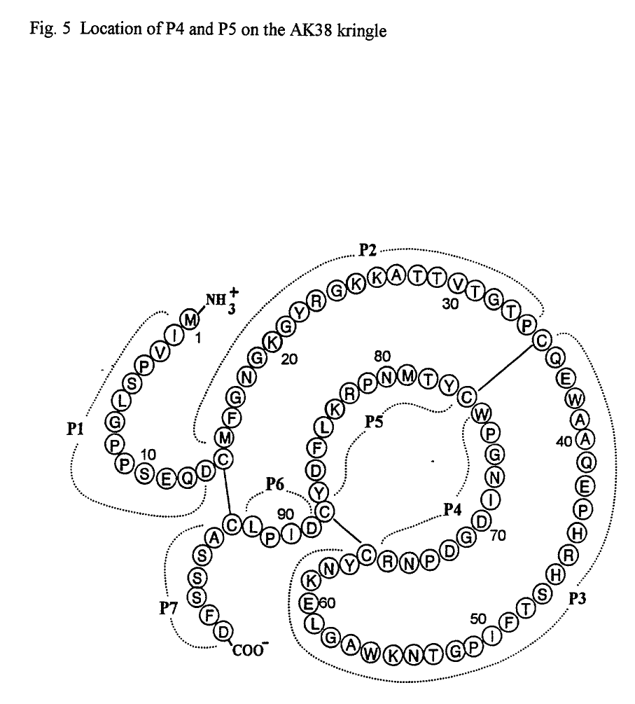

There are 6 units of cysteine in Ak38 protein, having 3 disulfide bonds and form an inner ring and an outer ring as a typical kringle structure (FIG. 5). The surprising and interesting point is that P4 and P5 both are on the inner ring of the kringle structure, and the others (P1, P2, P3, P6, and P7) are all on the outer ring. This observation indicates that the active site of AK38 is on the inner ring and P4 and P5 are primarily responsible for the anti-angiogenice effects.

Said peptides were synthesized using solid-phase peptide synthesis methodology by Fmoc chemistry. The side chain of the amino acid was protected by Pmc(2,2,5,7,8-pentamethychroman-6-sulfonyl) for arginine; Trt(trityl) for asparagine, glutamine and histidine; tBu(tert-butyl) for serine, threonne and tyrosine; Boc(tert-butyloxycarbonyl) for lysine.

### EXAMPLE 1

### Synthesis of P1

A standard 25 micromoles scale of above titled peptide was prepared by using Applied Biosystems peptide synthesizer model 433A. Briefly, an amide peptide synthesis column was installed in the peptide synthesis column position, and the following synthetic steps were applied: (1) solvate the resin with DMF (N,N-dimethylformamide) for 5 minutes; (2) deblock Fmoc group form of the resin-bound amino acid using 20% piperidine in DMF for 15 minutes; (3) wash resin with DMF for about 5 minutes; (4) activate the first amino acid, Fmoc protected Asp(O-tBu) (25 micromole) with a 0.2 M solution of HBTU (O-benzotriazolyl-N,N,N′,N′-tetramethyluronium hexafluorophosphat, 25 μM and HOBT (1-hydroxybenzotriazole, 25 μM) in DMSO (dimethyl sulfoxide)-NMP (N-methylpyrrolidone) and a 0.4 M solution of diisopropylethylamine (50 μM) in DMSO- NMP; (5) Couple the second amino acid, Fmoc-Gln(Trt) to the resin-bound amino acid in DMF for 30 minutes; (6) wash with DMF for 5 minutes. Then repeat step (3) through (6) with following amino acids coupled at step (5): Fmoc-Glu(O-tBu), Fmoc-Ser(t-Bu), Fmoc-Pro, Fmoc-Pro, Fmoc-Gly, Fmoc-Leu, Fmoc-Ser(t-Bu), Fmoc-Pro, Fmoc-Val and Fmoc-Ile. Finally, the resin was washed with THF (tetrahydrofuran) for 5 minutes, then dried for 10 minutes with argon flowed nitrogen.

The peptide was cleaved with TFA (trifluoroacetic acid) in the freshly-prepared solution consisted of 50 μL thioanisole, 25 μL water, 25 μL ethanedithiol and 0.9 mL TFA. The solution was mixed with resin-bound peptide for 15 minutes at 0° C., then the cleavage reaction was continued at 23° C. for 2 hours. Then the resin was filtered off. The peptide in the filtrate was precipitated with 16 times (v/v) of cold diethy ether. The peptide was collected by centrifugation and washed with ether and dried and lyophilized.

### EXAMPLE 2

### Synthesis of P2

The titled peptide was synthesized using the synthetic sequence described in Example 1, and using Fmoc-Pro as the starting amino acid. The coupling order was: Fmoc-Thr(t-Bu), Fmoc-Gly, Fmoc-Thr(t-Bu), Fmoc-Val, Fmoc-Thr(t-Bu), Fmoc-Thr(t-Bu), Fmoc-Ala, Fmoc-Lys(Boc), Fmoc-Lys(Boc), Fmoc-Gly, Fmoc-Arg(Pmc), Fmoc-Tyr(t-Bu), Fmoc-Gly, Fmoc-Lys(Boc), Fmoc-Gly, Fmoc-Asn(Trt), Fmoc-Gly, Fmoc-Phe and Fmoc-Met. The incubation time for cleaving peptide from resin was 2.5 hours at ambient temperature.

### EXAMPLE 3

### Synthesis of P3

Peptide compound indicated above was synthesized using the synthetic steps described in Example 1, and using Fmoc-Tyr(t-Bu) as the starting amino acid. The coupling order was: Fmoc-Asn(Trt), Fmoc-Lys(Boc), Fmoc-Glu(O-tBu), Fmoc-Leu, Fmoc-Gly, Fmoc-Ala, Fmoc-Trp, Fmoc-Lys(Boc), Fmoc-Asn(Trt), Fmoc-Thr(t-Bu), Fmoc-Gly, Fmoc-Pro, Fmoc-Ile, Fmoc-Phe, Fmoc-Thr(t-Bu), Fmoc-Ser(t-Bu), Fmoc-His(trt), Fmoc-Arg(Pmc), Fmoc-His(Trt), Fmoc-Pro, Fmoc-Glu(O-tBu), Fmoc-Gln(Trt), Fmoc-Ala, Fmoc-Ala, Fmoc-Trp, Fmoc-Glu(O-tBu) and Fmoc-Gln(Trt). The incubation time for cleaving reaction was 2.5 hours at ambient temperature.

### EXAMPLE 4

### Synthesis of P4

The titled peptide was synthesized using the synthetic steps described in Example 1, and using Fmoc-Trp as the starting amino acid. The coupling order was: Fmoc-Pro, Fmoc-Gly, Fmoc-Asn(Trt), Fmoc-Ile, Fmoc-Asp(O-tBu), Pmoc-Gly, Pmoc-Asp(O-tBu), Pmoc-Pro, Pmoc-Asn(Trt) and Pmoc-Arg(Pmc). The incubation time for cleaving peptide from resin was 2.5 hours at ambient temperature.

### EXAMPLE 5

### Synthesis of P5

The titled peptide was synthesized using the synthetic steps described in Example 1, and using Fmoc-Tyr(t-Bu) as the starting amino acid. The coupling order was: Fmoc-Asp(O-tBu), Fmoc-Phe, Fmoc-Leu, Fmoc-Lys(Boc), Fmoc-Arg(Pmc), Fmoc-Pro, Fmoc-Asn(Trt), Fmoc-Met, Fmoc-Thr(t-Bu) and Fmoc-Tyr(t-Bu). The incubation time for cleaving peptide from resin was 2.5 hours at ambient temperature.

### EXAMPLE 6

### Synthesis of P6

The titled compound was synthesized using the synthetic steps described in Example 1, and using Fmoc-Leu as the starting amino acid. The coupling order was: Fmoc-Pro, Fmoc-Ile and Fmoc-Asp(O-tBu). The incubation time for cleaving peptide from resin was 2 hours at ambient temperature.

### EXAMPLE 7

### Synthesis of P7

The titled peptide was synthesized using the synthetic steps described in Example 1, and using Fmoc-Asp(O-tBu) as the starting amino acid. The coupling order was: Fmoc-Phe, Fmoc-Ser(t-Bu), Fmoc-Ser(t-Bu), Fmoc-Ser(t-Bu) and Fmoc-Ala. The incubation time for peptide cleaving from resin was 2 hours at ambient temperature.

### EXAMPLE 8

### Purification of Said Peptides

HPLC (high performance liquid chromatography) was employed to purify said peptides disclosed in the present invention. HPLC was done using C-18 reversed phase column (20 mm×250 mm) with a flow rate of 15 mL/min. The buffers used were Buffer A (0.1% TFA in water) and Buffer B (0.01% TFA in acetonitrile). A gradient of 100%: 0% (Buffer A: Buffer B) to 0% :100% (Buffer A: Buffer B) was performed in 45 minutes.

Analytical HPLC was done using a C-18 column (4.6 mm×250 mm). The flow rate was 1 mL/min. The same gradient and buffers were used as described above, except the run time was 20 minutes.

### EXAMPLE 9

### In-vitro Inhibition of Anti-angiogenic Peptides Disclosed in the Present Invention

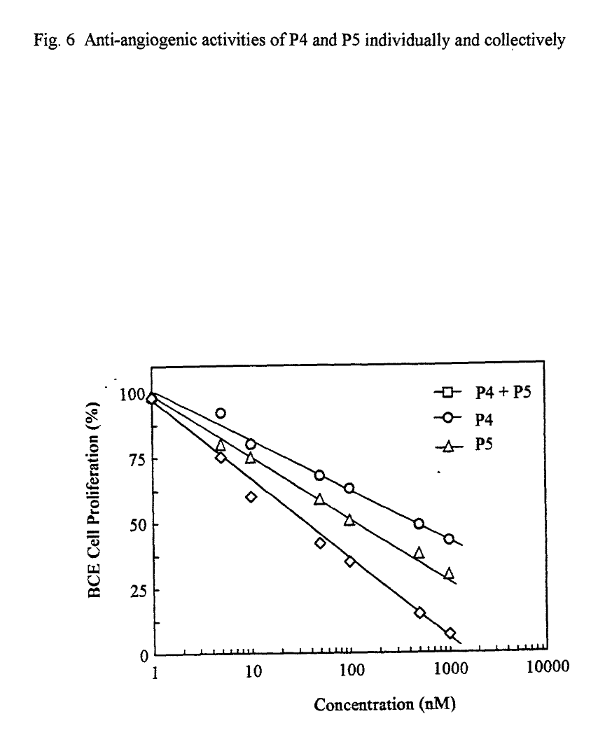

The inhibition of said peptide on the endothelial cells was performed on BCE cells (Folkman et al, Proceedings of the National Academy of Sciences USA. 76: 5217-5221 (1979)). The medium contains 90% DMEM (Dulbecco's Modified Eagle Medium), 10% FBS (fetal bovine serum), and 2 ng/mL of bFGF (basic fibroblast growth factor). A 24-well platelet was used for the growth of the BCE cells. To each well, 8,000-10,000 cells and 0.5 mL of the medium were added. The medium in each of the test wells was replaced by medium containing P1, P2, P3, P4, P5, P6, P7, or AK38 after 12 hours and continue to grow 5-7 days at 37° C. the BCE cells, in comparison to the control samples, to evaluate the inhibition on the growth of endothelial cells. The concentration inhibiting 50% of the cell growth is defined as IC₅₀. The IC₅₀ of P4 is 100 nM, and that of P5 is 45 nM. When P4 and P5 are used together, the inhibition was significantly stronger and the IC₅₀ becomes 30 nM indicating that there may be a synergistic effect between P4 and P5. (FIG. 6)

### EXAMPLE 10

### Synthesis of Related Peptides or their Derivatives

Using standard method for the synthesis of peptides, such as the method cited in the present invention, the following peptides or their derivatives may be made:

X-{4′}-Y (hereinafter {4=40 } is a peptide having the same amino acid sequence as that of {4}, but in the reversed order, i.e., the same amino acid sequence but the C-terminus becomes the N-terminus and the N-terminus becomes the C-terminus),

or

X-{5′}-Y (hereinafter {5′} is a peptide having the same amino acid sequence as that of {5}, but in the reversed order, i.e., the same amino acid sequence but the C-terminus becomes the N-terminus and the N-terminus becomes the C-terminus),

or

X-{4}-Ala-{5}-Y

or

X-{4′}-Ala-{5′}-Y

or

X-{5}-Ala-{4}-Y

or

X-{5′}-Ala-{4′}-Y

or

X-{4}-Cys-{5}-Y

or

X-{4′}-Cys-{5′}-Y

or

X-{5}-Cys- {4}-Y

or

X-{5′}-Cys-{4′}-Y

or

X-{4}-(Gly-Gly-Gly-Ser)ₙ-{5}-Y

or

X-{4′}-(Gly-Gly-Gly-Ser)ₙ-{5′}-Y

or

X-{5}-(Gly-Gly-Gly-Ser)ₙ-{4}-Y

or

X- {5}-(Gly-Gly-Gly-Ser)-ₙ-{4′}-Y

or

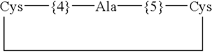

or

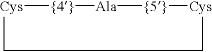

or

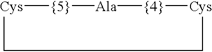

or

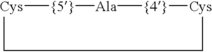

or

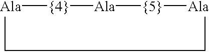

or

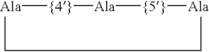

or

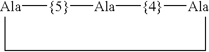

or

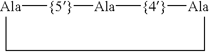

wherein X is Acetyl group or other customary N-terminal protecting groups; wherein Y is amine, ethylamine, or other customary C terminal protecting groups; wherein n=1 to 3.

### EXAMPLE 11

### Methods for Treating Cancer and Diseases Caused or Related to Angiogenesis

The following are some examples:

(10-1) Therapeutic amount of a pharmaceutical composition comprising P4 is administered to a patient of lung cancer for alleviating the disease.

(10-2) Therapeutic amount of a pharmaceutical composition comprising P5 is administered to a patient of brain cancer for alleviating the disease.

(10-3) Therapeutic amount of a pharmaceutical composition comprising P4 and P5 is administered to a patient of colon cancer for alleviating the disease.

(10-4) Therapeutic amount of a pharmaceutical composition comprising {4}-Ala-{5} is administered to a patient suffering blindness induced by late-staged diabetes.

(10-5) Therapeutic amount of a pharmaceutical composition comprising {4}-Cys-{5} is administered to a patient of lung cancer for alleviating the disease.

(10-6) Therapeutic amount of a pharmaceutical composition comprising {4′} is administered to a patient of lung cancer for alleviating the disease.

(10-7) Therapeutic amount of a pharmaceutical composition comprising {5′} is administered to a patient of lung cancer for alleviating the disease.

(10-8) Therapeutic amount of a pharmaceutical composition comprising

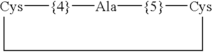

is administered to a patient of lung cancer for alleviating the disease.

(10-9) Therapeutic amount of a pharmaceutical composition comprising

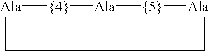

is administered to a patient of lung cancer for alleviating the disease. (10-10) Therapeutic amount of a pharmaceutical composition comprising

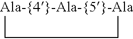

is administered to a patient a disease resulted from angiogenesis.

(10-11) Therapeutic amount of a pharmaceutical composition comprising a modified P5 is administered to a cancer patient wherein one or the amino acid in PS was in D-form.

(10-12) A pharmaceutical composition comprising P5 and customary excipient is administered by inhalation to a patient with brain cancer.

In summary, seven peptides were discovered based on the amino acid sequence of the kringle, AK38, on the Apolipoprotein. The present invention comprises, two anti-angiogenic peptides, their derivatives, and the method of treating cancer and other diseases related to angiogenesis. Although the description above contains many specificities, these should not be construed as limiting the scope of the invention but as merely providing the illustration of some of the presently preferred embodiments of this invention.

Thus, the scope of this invention should be determined by the appended claims and their legal equivalents, rather by examples given.

We claim:

1. A peptide or its derivative of the structure:
Arg-Asn-Pro-Asp-Gly-Asp-Ile-Asn-Gly-Pro-Trp (hereinafter referred as {4}),
or
Trp-Pro-Gly-Asn-Ile-Asp-Gly-Asp-Pro-Asn-Arg (hereinafter referred as {4′}),
or
Tyr-Thr-Met-Asn-Pro-Arg-Lys-Leu-Phe-Asp-Tyr (hereinafter referred as {5}),
or
Tyr-Asp-Phe-Leu-Lys-Arg-Pro-Asn-Met-Thr-Tyr (hereinafter referred as {5′}),
or
X-{4}-Y
or
X-{4′}-Y
or
X-{5}-Y
or
X-{5′}-Y
or
X-{4}-Ala-{5}-Y
or
X-{4′}-Ala-{5′}-Y
or
X-{5}-Ala-{4}-Y
or
X-{5}-Ala-{4}-Y
or
X-{4}-Cys-{5′}-Y
or
X-{4′}-Cys-{5′}-Y
or
X-{5}-Cys-{4}-Y
or
X-{5′}-Cys-{4′}-Y
or
X-{4}-(Gly-Gly-Gly-Ser)ₙ-{5}-Y
or
X-{4′}-(Gly-Gly-Gly-Ser)ₙ-{5′}-Y
or
X-{5}-(Gly-Gly-Gly-Ser)ₙ-{4}-Y
or 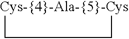
or 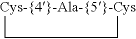
or 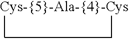
or 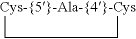
or 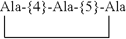
or 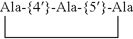
or 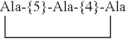
or 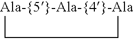
wherein the sequence of amino acids is written from the N-terminus (on the left) to the C-terminus (on the right);
wherein X is Acetyl group or other customary N-terminal protecting groups;
wherein Y is amine, ethylamine, or other customary C terminal protecting groups;
wherein n=1 to 3.

2. A pharmaceutical composition comprising one or a plurality of said peptide or its derivatives in claim 1.

3. Said peptide or its derivatives in claim 1 wherein one or a plurality of amino acids are in D-form.

4. A pharmaceutical composition comprising one or a plurality of said peptide or its derivatives in claim 3.

5. Said peptide or its derivatives in claim 1 wherein one amino acid is removed, replaced, or added to from a new molecule.

6. A pharmaceutical products comprising one or a plurality of said peptide or its derivatives in claim 5.

7. Said peptide or its derivatives in claim 1 wherein either the C-terminal protecting group or the N-terminal protecting group is removed if there is any.

8. A pharmaceutical product comprising one or a plurality of said peptide or its derivatives in claim 7.

9. A method for the treatment of diseases related to angiogenesis by administering to a patient a therapeutic amount of said peptides or its derivatives in claim 1.

10. The method in claim 9 wherein said diseases comprises lung cancer, liver cancer, brain cancer, colon cancer, impairment of vision induced by late-staged diabetes, and other diseases related to angiogenesis.

11. A method for the treatment of diseases related to angiogenesis by administering to a patient a pharmaceutical composition comprising of said peptides or its derivatives in claim 1

12. The method in claim 11 wherein said diseases comprises lung cancer, liver cancer, brain cancer, colon cancer, impairment of vision induced by late-staged diabetes, and other diseases related to angiogenesis.
